(19) United States
(12) Patent Application Publication
(10) Pub. No.: US 2024/0252461 A1
(43) Pub. Date: Aug. 1, 2024

(54) COMPOSITIONS AND METHODS FOR TREATMENT OF DEMYELINATION

(71) Applicant: Mordechai Chevion, MEVASERET ZION (IL)

(72) Inventors: Mordechai CHEVION, Jerusalem (IL); Vladimir VINOKUR, Beer Sheva (IL)

(21) Appl. No.: 17/047,169

(22) PCT Filed: Apr. 11, 2019
(86) PCT No.: PCT/IL2019/050412

§ 371 Date: Oct. 13, 2020

Related U.S. Application Data
(60) Provisional application No. 62/656,995, filed on Apr. 13, 2018.

(51) Int. Cl. A61K 31/315; A61K 38/03; A61P 37/06

(52) U.S. Cl. CPC A61K 31/315; A61K 31/16; A61K 38/02; A61P 37/06

(57) ABSTRACT

The present invention relates to a combination of a non-iron metal-desferrioxamine B complex and an immunomodulatory drug, for preventing, inhibiting, reducing or ameliorating demyelination, thus treatment of pathological conditions characterized by or associated with destruction and loss of myelin.

### TECHNICAL FIELD

The present invention relates to methods and compositions for treatment of the pathologic conditions characterized by destruction and loss of myelin.

### BACKGROUND ART

Central nervous system (CNS) demyelinating diseases are defined as disorders characterized by destruction and loss of myelin the fatty white substance that surrounds the nerve fiber, forming an electrically insulating layer (sheath) required for proper propagation of action potentials. A demyelinating disease is any condition that results in damage to the protective myelin covering the nerve fibers in the brain and spinal cord. When the myelin sheath is damaged, nerve impulses slow or even stop, causing neurological disorders. In demyelinating diseases, the pathological process takes place predominantly in the myelin sheath, while the axons remain relatively preserved. The demyelination process is mostly caused by inflammation, resulting from autoimmune response, metabolic derangements, focal compression, episodes of ischemia-reperfusion, or viral infection.

The most common demyelinating disorder of the CNS is multiple sclerosis (MS), which is an autoimmune disease. In this disorder, the immune system attacks the myelin sheath or the cells that produce and maintain it. This causes inflammation and injury to the sheath and ultimately to the nerve fibers that it surrounds, and may result in multiple areas of scarring (sclerosis).

Other types of demyelinating diseases include, e.g., optic neuritis, which results from inflammation of the optic nerve in one or both eyes: neuromyelitis optica (Devic's disease), which results from inflammation and demyelination of the central nervous system, especially of the optic nerve and spinal cord; transverse myelitis, which results from inflammation of the spinal cord; acute disseminated encephalomyelitis, which results from inflammation of the brain and spinal cord; and adrenoleukodystrophy and adrenomyeloneuropathy, which are rare, inherited metabolic disorders.

MS and other demyelinating diseases most commonly result in vision loss, muscle weakness, muscle stiffness and spasms, loss of coordination and sensation, pain, and changes in bladder and bowel function.

The pathologic hallmark of MS consists of areas of focal demyelination, known as plaques, characterized by variable gliosis, inflammation, and relative axonal preservation. Plaques location, as well as their number, size, and shape markedly vary among MS patients. Lesions are disseminated throughout the CNS, affecting both brain and spinal cord, but have a predilection for optic nerves, subpial region of the spinal cord, brainstem, cerebellum, and peri-ventricular white matter regions. Demyelinating disorders of the CNS include, e.g., neuromyelitis optica, Balo concentric sclerosis, and Schilder's disease.

Immune-mediated demyelinating diseases of the peripheral nervous system (PNS) were also characterized. Due to their markedly lower prevalence, data on the pathophysiology of demyelinating disorders of the PNS are considerably more limited. Examples of such disorders include, e.g., chronic inflammatory demyelinating polyneuropathy (CIDP) and Guillain-Barré syndrome (GBS).

Currently, there is no cure for MS and other CNS demyelinating diseases. For primary-progressive MS, ocrelizumab (Ocrevus®), the humanized anti-CD20 monoclonal antibody is the only Food and Drug Administration (FDA)-approved disease-modifying therapy. It slows worsening of disability in people with this type of MS. The primary aims of therapy are to restore myelin function after an attack (episode), prevent new attacks, and prevent disability. Much of the immune response associated with MS occurs in the early stages of the disease. Aggressive treatment with these medications as early as possible can lower the relapse rate and slow the formation of new lesions.

For relapsing-remitting MS, immunomodulators such as fingolimod (Gilenya®), dimethyl fumarate (Tecfidera®), teriflunomide (Aubagio®), glatiramer acetate (Copaxone®), and interferons-based medications constitute the first-line of treatment. Glatiramer acetate was shown to reduce relapses by approximately 30% (Hassan-Smith et al., 2011). On the other hand, La Mantia et al., 2010 discloses that glatiramer acetate “did not show any beneficial effect on the main outcome measures in MS, i.e., disease progression, and it does not substantially affect the risk of clinical relapses”. It is noteworthy that these medications show numerous adverse effects.

Glatiramer acetate is an immunomodulatory drug, administered subcutaneously daily or every other day, using a dose in the range of 20-40 mg per person. Glatiramer acetate is a random polymer of the four amino acids found in myelin basic protein, i.e., glutamic acid, lysine, alanine and tyrosine, and may work as a decoy for the immune system. It is approved for clinical use by the FDA for reducing the frequency of relapses, but not for reducing the progression of disability. The mechanism of action of glatiramer acetate is currently unknown; however, it was suggested that the administration of the drug shifts the population of T-cells from pro-inflammatory Th1 cells to regulatory Th2 cells, thereby suppressing the inflammatory response (Amon and Sela, 1999).

Furthermore, two additional humanized monoclonal antibodies, natalizumab (Tysabri®) and alemtuzumab (Lemtrada®), are available in clinics, although these medications are characterized by severe side effects and high costs.

Iron, a metallo-element abundant in mammalian tissues, including the human body, is an essential element for life, playing key roles in a variety of biological systems. In healthy adults, the total amount of iron is 3-4 g, of which about 1% is bound to iron-containing enzymes and redox-active proteins, including proteins involved in cellular respiration and electron transport.

“Labile iron pool” (LIP) is a small fraction of the total amount of iron. The LIP consists of labile and redox-active iron, which serves essential cellular purposes as well as the catalysis of production of reactive oxygen-derived species (ROS), including free radicals such as the hydroxyl radicals. ROS are known to generate oxidative stress, and to cause tissue injury and inflammation.

Accumulation of labile iron and oxidative stress were reported to be strongly linked to demyelinating disorders, including in MS (Mahad el al., 2015; Bagnato el al. 2013). For minimizing the injurious effects mediated by LIP and reducing LIP content in the brain, chelation therapy of iron has been proposed.

The most widely used iron chelating drug is Desferal®, which is the mesylate salt of desferrioxamine B (DFO). DFO is a siderophore, i.e., a small molecule with high-affinity for ferric iron, which is secreted by microorganisms and serves as a scavenger for environmental iron and as a shuttle for the importation of iron into the microbial cells. DFO is synthesized by the generally recognized as safe (GRAS) actinobacteria Streptomyces pilosus. Desferal® was developed by Ciba Geigy as a medication for clearance of iron overload, and was approved by the FDA for clinical use in 1964. Due to the large amounts of iron deposited within different tissues of hemochromatotic patients, and the low solubility of Desferal® in lipid phase, daily doses of >4000 mg/day/person were and still are being administered in patients. Structurally, DFO is a long, linear, hydrophilic molecule, which slowly and sparingly penetrates cell membranes, and barely enters tissues. Therefore, routes of Desferal® administration are limited to intramuscular, subcutancous, and intravenous injections only.

To overcome the limitations of the clinical use of Desferal®, described above, “non-iron” metal-ion complexes of DFO, such as zinc and gallium complexes of DFO, were prepared (U.S. Pat. Nos. 5,075,469 and 5,618,838). These complexes were found to be more effective than Desferal® alone in treatment of iron-mediated cell and tissue injury.

Administration of 2-5 mg/kg of Zn-DFO complex yielded high protection of the optic nerve, following prolonged ischemia and reperfusion (Obolensky et al., 2011). These DFO complexes showed enhanced ability to cross the blood-brain barrier. Also, the treatment of various conditions with either of these complexes did not result in any adverse effects in animal models.

Currently available treatment options for MS include a line of immunomodulatory drugs and humanized monoclonal antibodies, suppressing the pro-inflammatory activity resulting from different aspects. The mode of action of these drugs includes intervention in activation of the inflammatory cells, reduction of their ability to reach the site of inflammation, or targeting these cells for destruction. Yet, these drugs demonstrate limited efficacy and various adverse reactions of varying severity.

### SUMMARY OF INVENTION

In one aspect, the present invention relates to a method for preventing, inhibiting, reducing or ameliorating demyelination in a subject in need thereof, thereby more specifically treating a disease, disorder or condition characterized by or associated with demyelination, said method comprising administering to said subject a therapeutically effective amount of a combination comprising a metal-desferrioxamine B complex (metal-DFO complex) or a pharmaceutically acceptable salt thereof, wherein said metal is not iron, and an immunomodulatory drug. In certain embodiments, the combination administered according this method comprises a sub-therapeutic dose of said immunomodulatory drug.

In another aspect, the present invention provides a pharmaceutical composition comprising a combination of a metal-DFO complex or a pharmaceutically acceptable salt thereof, wherein said metal is not iron, and an immunomodulatory drug, and a pharmaceutically acceptable carrier. Such a pharmaceutical composition is useful in preventing, inhibiting, reducing or ameliorating demyelination, thereby more specifically treating a disease, disorder or condition characterized by or associated with demyelination.

In still another aspect, the present invention relates to a combination of a metal-DFO complex or a pharmaceutically acceptable salt thereof, wherein said metal is not iron, and an immunomodulatory drug, for use in preventing, inhibiting, reducing, or ameliorating demyelination.

In yet another aspect, the present invention relates to use of a combination of a metal-DFO complex or a pharmaceutically acceptable salt thereof, wherein said metal is not iron, and an immunomodulatory drug in the preparation of a pharmaceutical composition for preventing, inhibiting, reducing, or ameliorating demyelination.

In a further aspect, the present invention provides a kit comprising:
(i) either a pharmaceutical composition A comprising a metal-DFO complex or a pharmaceutically acceptable salt thereof; or pharmaceutical compositions B and C, wherein pharmaceutical composition B comprises DFO or a pharmaceutically acceptable salt thereof, and pharmaceutical composition C comprises ions of a metal, wherein said metal is not iron;
(ii) a pharmaceutical composition D comprising an immunomodulatory drug; and
(iii) instructions to administer either (a) pharmaceutical compositions A and D, either concomitantly or sequentially at any order and within a time period not exceeding 36 hours; or (b) pharmaceutical compositions B, C and D, cither concomitantly or sequentially at any order and within a time period not exceeding 36 hours, so as to form in situ, upon complexation of said DFO or pharmaceutically acceptable salt thereof and said metal ions, a metal-DFO complex or a pharmaceutically acceptable salt thereof, to thereby prevent, inhibit, reduce, or ameliorate demyelination.

### DETAILED DESCRIPTION OF THE INVENTION

The term “DFO”, “deferoxamine” or “desferrioxamine B”, used herein interchangeably, refers to the compound N′-[5-(acetyl-hydroxy-amino)pentyl]-N-[5-[3-(5-aminopentyl-hydroxy-carbamoyl) propanoylamino]pentyl]-N-hydroxy-butane diamide, a bacterial siderophore, which is made up from six basic units and naturally produced by the actinobacteria Streptomyces pilosus. When not bound to a metal, DFO is a linear, noodle-like, molecule that sparingly infiltrates into cells; however, upon metal binding it becomes less polar and assumes a globular complex capable of infiltrating into cells. These considerations explain why the DFO complexes more easily penetrate through cellular membranes, and more effectively bind intracellular iron that is redox active and mediates tissue damage.

It is postulated that some of the useful effects exerted by DFO in inhibiting ROS formation are achieved through its actions as a chelating agent of ferric iron (chelant, chelator, or sequestering agent). In addition, DFO is capable of forming soluble complexes, i.e., chelates, with certain (non-iron) metal ions, that are redox-inactive and consequently they cannot normally react with other elements or ions. Such chelates often have chemical and biological properties that are markedly different from those of either the chelator or the metal ion, alone.

In addition to iron, DFO forms a tight complex with redox-silent metals such as zinc and gallium. In recent experiments comparing the ability of DFO alone and the Zn-DFO complex to infiltrate into cells in a tissue culture model using H9C2 cardiomyocytes, it has been found that the Zn-DFO complex infiltrates into the cells more than three-fold faster than DFO alone (data not shown). Using the zinc (or a different non-iron metal) complex of DFO may thus provide two-step antioxidant protection, wherein the redox-active iron is chelated and its redox activity is arrested; and the zinc that had been a part of the DFO complex, which in itself possesses antioxidant activity and is needed for the adequate functioning of various enzymes, or the other non-iron metal, is then released in a controlled manner.

Desferal® is a commercially available DFO marketed in the form of its methanesulfonate (mesylate) salt. Other pharmaceutically acceptable salts of DFO include, without being limited to, the chloride, bromide, iodide, acetate, ethanesulfonate (esylate), ethanedisulfonate (edisylate), maleate, fumarate, tartrate, bitartrate, sulfate, p-toluenesulfonate, benzenesulfonate, tosylate, benzoate, acetate, phosphate, carbonate, bicarbonate, succinate, and citrate salt thereof.

The relative stability constants for the DFO complexes with Fe(III), Cu(II), Zn(II) and Ga(III) are 1031, 1014, 1011 and 1028, respectively (Keberle, 1964). The stability constant of a DFO complex with a lanthanide ions is expected to be lower than 1031 (Orcutt et al., 2010). Based on these thermodynamic properties, upon penetration into cells, with high abundance of labile and redox-active Fe, the Zn-DFO complex exchanges the Zn with Fe, and the zinc released from the complex could have an additional beneficial antioxidant and/or other effects. For instance, MS pathophysiology is also shown to involve Zn loss (Popescu et al., 2017), so replenishment of Zn in the brain can also serve as an additional beneficial factor.

As has been shown, iron-binding through its exchange with the non-iron metal-DFO complexes, such as the zinc ion within the Zn-DFO complex and the gallium ion within the Ga-DFO complex, demonstrate a significant anti-oxidant and anti-inflammatory potential in various models of inflammatory disorders (Obolensky et al., 2011; Bibi et al., 2014; Morad et al., 2005), preventing formation of ROS catalyzed by labile iron, and suppressing the production of pro-inflammatory cytokines. These complexes were further found completely safe and efficacious in alleviation of the ischemia-reperfusion injury (Karck et al., 2001), which was reported to contribute to demyelination process (Renner et al., 2017). Furthermore, these complexes demonstrated an ability to infiltrate the blood-brain barrier (BBB) notably better than DFO alone. As further demonstrated in the experimental section herein, Zn-DFO and Ga-DFO complexes are highly effective in diminishing the rate of a demyelination process, i.e., inhibiting, reducing or ameliorating demyelination, thus capable of treating medical conditions associated with or characterized by demyelination. The treatment of plethora of pathologies with either of these complexes did not result in any adverse effects in the animal models.

Importantly, the Fe-DFO complex, resulting from the exchange of zinc of Zn-DFO complex or gallium of Ga-DFO complex by tissue-borne iron, is an inert complex where the iron is not involved in redox cycling, and is excreted out of the body.

The therapeutic concept underlying the present invention is the use of a combination comprising, or consisting of, a non-iron metal-DFO complex (herein also referred to a “Zygosid”), eg., Zn-DFO, Ga-DFO, or a mixture thereof, and an immunomodulatory drug, for preventing, inhibiting, reducing or ameliorating demyelination, and consequently treating a disease, disorder or condition characterized by or associated with demyelination. Demyelination is caused mostly by inflammation resulting from an autoimmune response, and Zygosids have already demonstrated in several model systems, including of autoimmune disorders such as diabetes and psoriasis, a potent anti-inflammatory activity without noticeable adverse side effects. The combination disclosed, when administered to a subject suffering from a disease, disorder or condition characterized by or associated with demyelination, is thus expected to have a therapeutic efficacy that is substantially higher than that of each one of the drugs alone, and even in cases wherein the dosage of each one of the active agents, when administered alone, has either very low therapeutic effect or no such effect at all.

It is postulated that the protective effect of the metal-DFO complex stems from more than one reason. The first is the suppressed formation of ROS. The ability of the metal-DFO complex to act via a combined “push-pull” mechanism to achieve such a marked reduction in free radical formation is supported by both theoretical considerations and previously reported experimental findings. In the Fenton reaction or in the metal-mediated Haber-Weiss mechanism, the conversion of low reactive species to the highly reactive hydroxyl radicals apparently depends on the availability of trace amounts of redox-active and labile iron or copper ions, which serve as essential catalysts in ROS formation (Chevion, 1988: Samuni et al., 1983). It is thus hypothesized that said complex, and particularly Zn-DFO and Ga-DFO complexes, exert their protective effect by intervening in this critical step of hydroxyl radical formation. The parallel mechanism of action is the removal of iron, which is directly incriminated in accelerating demyelination (Hametner et al., 2018).

In one aspect, the present invention thus relates to a method for preventing, inhibiting, reducing or ameliorating demyelination, thereby more specifically treating a disease, disorder or condition characterized by or associated with demyelination, in a subject in need thereof, said method comprising administering to said subject a therapeutically effective amount of a combination comprising a metal-DFO complex or a pharmaceutically acceptable salt thereof, wherein said metal is not iron (herein also referred to as a “non-iron metal-DFO complex”), and an immunomodulatory drug.

In certain embodiments, the non-iron metal-DFO complex administered according to the method of the present invention is the zinc-DFO complex, gallium-DFO complex, manganese-DFO complex, copper-DFO complex, aluminum-DFO complex, vanadium-DFO complex, indium-DFO complex, chromium-DFO complex, gold-DFO complex, silver-DFO complex, or platinum-DFO complex, a lanthanide-DFO complex, or a mixture thereof. Particular lanthanides include lanthanum, cerium, praseodymium, neodymium, promethium, samarium, europium, gadolinium, terbium, dysprosium, holmium, erbium, thulium, ytterbium, and lutetium, of which europium and gadolinium are preferred. According to the invention, in cases wherein a mixture of two or more metal-DFO complexes is administered, said mixture may comprise said metal-DFO complexes in any quantitative ratio. For example, in case a mixture of two metal-DFO complexes is administered, said mixture may comprise said two metal-DFO complexes in a quantitative ratio of about 100:1 to about 1:100, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100. Similarly, in case a mixture of three metal-DFO complexes is administered, said mixture may comprise said three metal-DFO complexes in a quantitative ratio of, e.g., about 1:1:1, about 1:2:3, about 1:10:50, about 1:20:50, about 1:10:100, or about 1:50:100.

In particular embodiments, the non-iron metal-DFO complex administered according to the method of the present invention is Zn-DFO complex, Ga-DFO complex, or a mixture of Zn-DFO complex and any one of the other non-iron metal-DFO complexes listed above, e.g., Ga-DFO complex. In more particular such embodiments, a mixture of Zn-DFO complex and an additional metal-DFO complex, e.g., Ga-DFO complex, is administered, e.g., wherein the quantitative ratio of the Zn-DFO complex to the other metal-DFO complex is in a range of about 100:1 to about 1:100, e.g., about 50:1 to about 1:50, about 40:1 to about 1:40, about 30:1 to about 1:30, about 20:1 to about 1:20, about 10:1 to about 1:10, about 5:1 to about 1:5, about 4:1 to about 1:4, about 3:1 to about 1:3, about 2:1 to about 1:2, or about 1:1. Certain such mixtures are those wherein the amount of the Zn-DFO complex is higher than that of the other metal-DFO complex, e.g., mixtures wherein the quantitative ratio of the Zn-DFO complex to the other metal-DFO complex is in a range of about 10:1 to about 2:1, e.g., about 10:1, about 9:1, about 8:1, about 7:1, about 6:1, about 5:1, about 4:1, about 3:1, or about 2:1. Other such mixtures are those wherein the amount of the Zn-DFO complex is lower than that of the other metal-DFO complex, e.g., mixtures wherein the quantitative ratio of the Zn-DFO complex to the other metal-DFO complex is in a range of about 1:2 to about 1:10, e.g., about 1:2, about 1:3, about 1:4, about 1:5, about 1:6, about 1:7, about 1:8, about 1:9, or about 1:10.

The term “immunomodulatory drug” as use herein refers to an active agent capable of modifying the immune response or the functioning of the immune system by either the stimulation of antibody formation or the inhibition of white blood cell activity.

According to the method of the present invention, the immunomodulatory drug administered in combination with the metal-DFO complex may be fingolimod, dimethyl fumarate, teriflunomide, glatiramer acetate, ocrelizumab, natalizumab, alemtuzumab, an immuno-enhancing interferon-based drug, or a pharmaceutically acceptable salt thereof.

In certain embodiments, the immunomodulatory drug administered according to the method of the invention is fingolimod, or a pharmaceutically acceptable salt thereof. Fingolimod is a sphingosine-1 phosphate receptor modulator, which sequesters lymphocytes in lymph nodes, preventing them from contributing to an autoimmune reaction. A particular such drug is Gilenya®, currently used for treating relapsing forms of MS in patients 10 years of age and older. Gilenya® is available as capsules containing 0.25 mg or 0.5 mg fingolimod (as its hydrochloride salt). The recommended dosage is 0.5 mg or 0.25 mg orally once a day, depending on the weight of the subject treated. Gilenya® doses higher than 0.5 mg are associated with a greater incidence of adverse reactions without additional benefit.

In other embodiments, the immunomodulatory drug administered according to the method of the invention is dimethyl fumarate or a pharmaceutically acceptable salt thereof, such as Tecfidera® currently used for treating relapsing forms of MS. According to the dosing information available for this drug, the starting dose is 120 mg, taken orally twice a day, which increases after 7 days to a maintenance dose of 240 mg twice a day. Temporary dose reductions to 120 mg twice a day may be considered for individuals who do not tolerate the maintenance dose; and within 4 weeks, the recommended dose of 240 mg twice a day should be resumed,

In further embodiments, the immunomodulatory drug administered according to the method of the invention is teriflunomide, or a pharmaceutically acceptable salt thereof which inhibits the mitochondrial enzyme dihydroorotate dehydrogenase that is involved in the de novo synthesis of pyrimidine. A particular such drug is Aubagio®, which is formulated as film-coated tablets for oral administration containing 7 mg or 14 mg teriflunomide, wherein the recommended dose is one tablet once a day.

In yet further embodiments, the immunomodulatory drug administered according to the method of the invention is glatiramer acetate, which is a mixture of random-sized peptides composed of the four amino acids found in myelin basic protein, i.e., glutamic acid, lysine, alanine and tyrosine. Myelin basic protein is the antigen in the myelin sheaths of the neurons that stimulates an autoimmune reaction in people with MS, so the peptide may work as a decoy for the attacking immune cells. Glatiramer acetate is approved to reduce the frequency of relapses, but not for reducing the progression of disability. Observational studies, but not randomized controlled trials, suggest that it may reduce progression of disability. While a conclusive diagnosis of MS requires a history of two or more episodes of symptoms and signs, glatiramer acetate is approved to treat a first episode anticipating a diagnosis. A particular such drug is Copaxone®, currently used for treating MS including relapsing-remitting MS. Copaxone® is approved as either a daily 20 mg injection or 40-mg dose injected three-times weekly. Glatopa® is a generic version of Copaxone®, approved for the same indication and at the same dosages.

In yet other embodiments, the immunomodulatory drug administered according to the method of the invention is ocrelizumab or a pharmaceutically acceptable salt thereof, such as Ocrevus®, which is a humanized monoclonal antibody approved for treatment of both relapsing MS and primary progressive MS (PPMS). The recommended initial dose for Ocrevus® is 600 mg intravenously injected 2 weeks apart (300 mg in each injection), and a subsequent dose of 600 mg intravenous infusion every six months.

In still further embodiments, the immunomodulatory drug administered according to the method of the invention is natalizumab or a pharmaceutically acceptable salt thereof, such as Tysabrik; or alemtuzumab or a pharmaceutically acceptable salt thereof, such as Lemtrada® or Campath®, both humanized monoclonal antibodies. Natalizumab is believed to work by reducing the ability of inflammatory immune cells to attach to and pass through the cell layers lining the intestines and blood-brain barrier. Due to the lack of information about the long-term use of Natalizumab, as well as potentially fatal adverse events, reservations have been expressed over the use of the drug outside of comparative research with existing medications. The recommended does is 300 mg intravenous infusion over 1 hour once every 4 weeks. Alemtuzumab, owing to its safety profile, is used in MS patients who have inadequate response to two or more of the other drugs. The recommended dose is administered as two separate treatment courses: (i) 12 mg/day intravenous injection during 5 consecutive days (60 mg total does); and (ii) 12 mg/day intravenous injection during 3 consecutive days (36 mg total does). Subsequent courses include administration of 12 mg/day intravenously during 3 consecutive days (36 mg total dose), as needed, at least 12 months after last does of any prior treatment course.

Interferons are cytokines, i.e., small proteins that are involved in intercellular signaling, wherein the three forms alpha, beta, and gamma control the activity of the immune system. Interferon-alpha is produced by white blood cells other than lymphocytes, interferon-beta is produced by fibroblasts, and interferon-gamma is produced by natural killer cells and cytotoxic T lymphocytes. Interferon alpha and beta are classified as type I interferons, which mainly induce viral resistance in cells; and interferon gamma is classified as type II, which mainly signals the immune system to respond to infectious agents or cancerous growth.

Interferon beta-based medications are used to treat MS. Such medications reduce the frequency of exacerbations, stabilize the course of the disease, and may also slow down the worsening of symptoms and help people have less physical disability over time. There are five types of commercially available interferon beta-based drugs, all of which are injectable, which are classified into interferon-beta 1a drugs, i.e., Avonex®, Rebif® and Plegridy®; and interferon-beta 1b drugs, i.e., Betaferon® and Extavia®. The recommended dosage of (i) Avonex® is 30 μg intramuscular injected once a week: (ii) Rebif® is 22 μg or 44 μg subcutaneously injected 3 times a week; and (iii) Plegridy® is 125 μg injected subcutaneously every 14 days. The interferon-beta 1b drugs are subcutaneously administered at an initial dosage of 0.0625 mg every other day, which is increased (in 25% increments) every 2 weeks, over a 6-week period, to a maintenance dose of 0.25 mg every other day.

According to the method of the present invention, the non-iron metal-DFO complex and immunomodulatory drug administered can be at any quantitative ratio. In certain embodiments, the quantitative ratio of said metal-DFO complex to said immunomodulatory drug in said combination is in a range of 100:1 to 1:100, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100.

According to the method disclosed herein, the metal-DFO complex administered to the subject treated along with the immunomodulatory drug may enable decreasing the dosage of said immunomodulatory drug to a sub-therapeutic dose, i.e., a dose that is insufficient for producing a therapeutic effect when administered alone, as well as increasing the time intervals between consecutive treatments utilizing said immunomodulatory drug.

In certain embodiments, the combination administered according to the method of the present invention as defined in any one of the embodiments above, comprises a sub-therapeutic dose of the immunomodulatory drug. Such a combination may lead to reduced adverse effects compared to the adverse effects caused by said immunomodulatory drug when it is administered alone at a therapeutically effective dose.

In particular such embodiments, the combination administered according to the invention comprises a non-iron metal-DFO complex, e.g., Za-DFO complex, Ga-DFO complex, or a mixture thereof (for example, wherein the quantitative ratio of said Zn-DFO complex to said Ga-DFO complex is in a range of 100:1 to 1:100), and an immunomodulatory drug selected from fingolimod, dimethyl fumarate, teriflunomide, glatiramer acetate, ocrelizumab, natalizumab, alemtuzumab, an immune-enhancing interferon-based drug, or a pharmaceutically acceptable salt thereof, at a sub-therapeutic dose. More particular such embodiments are those wherein the non-iron metal-DFO complex is as defined above, and the immunomodulatory drug is (i) Gilenya®; (ii) Tecfidera®; (iii) Aubagio®; Copaxone®; (iv) Glatopa®; (v) Ocrevus®; (vi) Tysabri®; (vii) Lemtrada®; or (viii) Campath®.

In certain embodiments, the non-iron metal-DFO complex and immunomodulatory drug administered according to the method of the present invention, as defined in any one of the embodiments above, are formulated as separate, e.g., two, pharmaceutical compositions for administration either concomitantly or sequentially at any order and within a time period not exceeding 36 hours, through one or more routes of administration. According to the method of the invention, each one of the compositions administered may be independently formulated for any suitable administration route, e.g., for oral, sublingual, buccal, rectal, intravenous, intraarterial, intramuscular, intraperitoneal, intrathecal, intrapleural, intratracheal, cutaneous, subcutancous, transdermal, intradermal, nasal, vaginal, ocular, otic, or topical administration, or for inhalation.

In other embodiments, the non-iron metal-DFO complex and immunomodulatory drug administered according to the method of the present invention, as defined in any one of the embodiments above, are formulated as a sole pharmaceutical composition. Such a composition may be formulated for any suitable administration route, e.g., for oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

The method disclosed herein, according to any one of the embodiments defined above, is aimed at preventing, inhibiting, reducing or ameliorating demyelination, thereby treating a disease, disorder or condition characterized by or associated with demyelination in a subject in need thereof, by administering a therapeutically effective amount of a drug combination, also referred to herein as active agent combination, comprising a non-iron metal-DFO complex or a pharmaceutically acceptable salt thereof, and an immunomodulatory drug.

The term “demyelination”, also referred to as “demyelination disease”, is a pathologic process occurring in the nervous system in which the myelin sheath is damaged. Myelin is a fatty substance formed in the CNS by glial cells called oligodendrocytes, and in the PNS by Schwann cells. The damage to the myelin sheath impairs the conduction of signals in the affected nerves. Consequently, the reduction in conduction ability causes deficiency in sensation, movement, cognition, and/or other functions depending on which nerves are involved. The demyelination process might be due to genetics, infectious agents, autoimmune reactions, as well as unknown factors. Depending on the primary site of demyelination in the nervous system, these diseases are divided into central demyelination involving the CNS, and peripheral demyelination affecting the PNS. Demyelinating diseases may also be divided, according to the presence or lack of inflammation, to inflammatory diseases, and may be further divided depending on the underlying reason for demyelination, to myclino-clastic diseases, wherein a normal and healthy myelin is destroyed by a toxic, chemical, or autoimmune substance; and demyelinating leukodystrophic diseases, wherein the myelin is abnormal and degenerates.

The term “subject” as used herein refers to any mammal, e.g., a human, nonhuman primate, horse, ferret, dog, cat, cow, and goat. In a preferred embodiment, the term “subject” denotes a human, i.e., an individual.

The term “treatment” as used herein with respect to a disease, disorder or condition characterized by or associated with demyelination, refers to the administration of a therapeutically effective amount of a drug combination as described above, which is effective to ameliorate undesired symptoms associated with said disease, disorder or condition: prevent the manifestation of such symptoms before they occur: slow down the progression of said disease, disorder or condition: slow down the deterioration of symptoms: enhance the onset of remission period: slow down the irreversible damage caused in the progressive chronic stage of said disease, disorder or condition; delay the onset of said progressive stage: lessen the severity or cure said disease, disorder or condition: improve survival rate or more rapid recovery; and/or prevent said disease, disorder or condition form occurring.

The term “therapeutically effective amount” as used herein with respect to the drug combination administered according to the method of the invention refers to an amount of said drug combination, more particularly amounts of said metal-DFO complex and said immunomodulatory drug, that upon administration under a particular regimen during a particular period of time, e.g., days, weeks, months or years, is sufficient to prevent, inhibit, reduce or ameliorate an demyelination occurring in the body of the subject administered with. The actual dosages of both the metal-DFO complex and the immunomodulatory drug administered may be varied so as to obtain amounts of said metal-DFO complex and said immunomodulatory drug that are effective to achieve the desired prophylactic/therapeutic response for a particular subject and mode of administration, without being toxic to the subject. The dosage level selected will depend upon a variety of factors including the activity of the metal-DFO complex employed, the route of administration, the duration of the treatment, and other drugs, if any, used in combination with the drug combination employed, as well as the age, sex and weight of the subject treated, and the severity/progression of the medical condition. In general, it may be presumed that for preventive treatment, lower doses will be needed, while higher doses will be required for treatment of subjects already showing pathological phenotypes of said demyelination. The term “sub-therapeutic dose” as used herein with respect to the immunomodulatory drug composing the drug combination refers to a daily dose of said immunomodulatory drug that is lower than that sufficient to prevent, inhibit, reduce, or ameliorate demyelination in a subject in need thereof, when administered to said subject alone (i.e., without said non-iron metal-DFO complex), under a particular regimen and during a particular time period.

In certain embodiments, the disease, disorder or condition characterized by or associated with demyelination and thus treated by the method of the present invention include, without limiting, multiple sclerosis, neuromyelitis optica (Devic's disease), Balo concentric sclerosis, Schilder's disease, chronic inflammatory demyelinating polyneuropathy, progressive multifocal leukoencephalopathy, Guillain-Barré syndrome, progressive inflammatory neuropathy, acute disseminated encephalomyelitis, optic neuritis, transverse myelitis, adrenoleukodystrophy, and adrenomyeloneuropathy.

In other embodiments, the disorder or condition characterized by or associated with demyelination and thus treated by the method of the present invention is induced by an injury, such as an injury caused by a mechanical force, ischemia, a toxic agent such as a herbicide or pesticide, or hemorrhage.

In another aspect, the present invention provides a pharmaceutical composition comprising a drug combination as defined above, i.e., a combination of a non-iron metal-DFO complex or a pharmaceutically acceptable salt thereof, and an immunomodulatory drug, and a pharmaceutically acceptable carrier.

The drug combination comprised within the pharmaceutical composition of the present invention may be any combination of a non-iron metal-DFO complex or a pharmaceutically acceptable salt thereof, and an immunomodulatory drug.

In certain embodiments, the non-iron metal-DFO complex comprised within the pharmaceutical composition of the invention is the zinc-DFO complex, gallium-DFO complex, manganese-DFO complex, copper-DFO complex, aluminum-DFO complex, vanadium-DFO complex, indium-DFO complex, chromium-DFO complex, gold-DFO complex, silver-DFO complex, or platinum-DFO complex, a lanthanide-DFO complex, or a mixture thereof. Mixtures of metal-DFO complexes, when used, may comprise two metal-DFO complexes in any quantitative ratio, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100. Other mixtures may comprise three metal-DFO complexes in any quantitative ratio, e.g., in a quantitative ratio of, e.g., about 1:1:1, about 1:2:3, about 1:10:50, about 1:20:50, about 1:10:100, or about 1:50:100.

In particular embodiments, the metal-DFO complex comprised within the pharmaceutical composition of the invention is Zn-DFO complex, Ga-DFO complex, or a mixture of Zn-DFO complex and any one of the other metal-DFO complex listed above, e.g., Ga-DFO complex. In more particular such embodiments, a mixture of Zn-DFO complex and an additional metal-DFO complex, e.g., Ga-DFO complex, is used, e.g., wherein the quantitative ratio of the Zn-DFO complex to the other metal-DFO complex is in a range of about 100:1 to about 1:100, e.g., about 50:1 to about 1:50, about 40:1 to about 1:40, about 30:1 to about 1:30, about 20:1 to about 1:20, about 10:1 to about 1:10, about 5:1 to about 1:5, about 4:1 to about 1:4, about 3:1 to about 1:3, about 2:1 to about 1:2, or about 1:1. Certain such mixtures are those wherein the amount of the Zn-DFO complex is higher than that of the other metal-DFO complex, e.g., mixtures wherein the quantitative ratio of the Zn-DFO complex to the other metal-DFO complex is in a range of about 10:1 to about 2:1, e.g., about 10:1, about 9:1, about 8:1, about 7:1, about 6:1, about 5:1, about 4:1, about 3:1, or about 2:1. Other such mixtures are those wherein the amount of the Zn-DFO complex is lower than that of the other metal-DFO complex, e.g., mixtures wherein the quantitative ratio of the Zn-DFO complex to the other metal-DFO complex is in a range of about 1:2 to about 1:10, e.g., about 1:2, about 1:3, about 1:4, about 1:5, about 1:6, about 1:7, about 1:8, about 1:9, or about 1:10.

In certain embodiments, the immunomodulatory drug comprised within the pharmaceutical composition of the invention is fingolimod, dimethyl fumarate, teriflunomide, glatiramer acetate, ocrelizumab, natalizumab, alemtuzumab, an immuno-enhancing interferon-based drug, or a pharmaceutically acceptable salt thereof, as referred to above.

The drug combination comprised within the pharmaceutical composition of the present invention may contain the non-iron metal-DFO complex and the immunomodulatory drug at any quantitative ratio. In certain embodiments, the quantitative ratio of said metal-DFO complex to said immunomodulatory drug in the drug combination is in a range of 100:1 to 1:100, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100.

The metal-DFO complexes for use according to the method and composition of the present invention may be prepared utilizing any technology or procedure known in the art, e.g., as described in International Publication No. WO2011021203. Possible procedures for the preparation of Zn-DFO and Ga-DFO complexes having particular metal:DFO stoichiometric ratios are provided herein below. Other such complexes having different metal:DFO stoichiometric ratios may be prepared using similar procedures.

A Zn-DFO complex having Zn:DFO stoichiometric ratio of 1.0:1.0 may be prepared, e.g., by mixing 10 mM solution of DFO with an equal volume of 10 mM ZnCF₂ solution, titrating to a pH between 5.0 to 7.5, heating the mixture to 45° C. for 30 min, and cooling down. Alternatively, such a complex may be prepared by drying the contents of 1 vial (500 mg, 0.76 mmole) of Desferal®, by adding 168 mg of dry zinc acetate anhydrous (0.76 mmole), adding double distilled water until the contents fully dissolve (about 10 ml), warming the solution to 40° C. for 45 minutes, and cooling down.

A Zn-DFO complex having Zn:DFO stoichiometric ratio of 1.0:1.25 may be prepared, e.g., by mixing 10 mM solution of DFO with an equal volume of 6 mM ZnCl₂ solution, titrating to a pH between 5.0 to 7.5, heating to 45° C. for 30 min, and cooling down.

A Zn-DFO complex having Zn:DFO stoichiometric ratio of 0.6:1.0 may be prepared, e.g., by mixing 10 mM DFO solution with an equal volume of 12.5 mM ZnCl₂ solution and 10 ml of 5.5 mM histidine, titrating to a pH between 5.0 to 7.5, heating to 45° C. for 30 min, and cooling down.

A Zn-DFO complex having Zn:DFO stoichiometric ratio of 0.2:1.0 may be prepared, e.g., by mixing 50 mM DFO solution with ⅕ the volume of 50 mM ZnSO₄ solution, at the same pH recited above, heating to 40° C. for 45 min, and cooling down.

A Ga-DFO complex having Ga:DFO stoichiometric ratio of 1.0:1.0 may be prepared, e.g., by mixing 10 mM solution of DFO with an equal volume of 10 mM GaCl₃ solution, titrating to pH of about 5.0 and then to a pH between 6.0 to 7.5 (using NaOH). A similar complex having Ga:DFO stoichiometric ratio of 0.6:1.0 may be prepared, e.g., by mixing 5 mM DFO solution with an equal volume of 3 mM GaCl₃ solution, titrating to a pH between 5.0 to 7.5.

Pharmaceutical compositions as disclosed herein may be prepared by conventional techniques, e.g., as described in Remington: The Science and Practice of Pharmacy, 19th Ed., 1995. The compositions can be prepared, e.g., by uniformly and intimately bringing the active agents, i.e., the non-iron metal-DFO complex(es) and the immunomodulatory drug, into association with a liquid carrier, a finely divided solid carrier, or both, and then, if necessary, shaping the product into the desired formulation. The active agents may be applied as is, or conjugated to one or more pharmaceutically acceptable groups such as sugars, starches, amino acids, polyethylene-glycol (PEG), polyglycerol-based compounds, hydrazines, hydroxylamines, amines, or halides. The compositions may be in the form of a liquid (e.g., solution, emulsion, or suspension), gel, cream, solid, semisolid, film, foam, lyophilisate, or aerosol, and may further include pharmaceutically and physiologically acceptable fillers, carriers, diluents or adjuvants, and other inert ingredients and excipients. In one embodiment, the pharmaceutical composition of the invention is formulated as nanoparticles or microparticles.

The pharmaceutical compositions of the present invention may be formulated for any suitable route of administration, e.g., oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

The pharmaceutical compositions of the invention, when formulated for oral administration, may be in any suitable form, e.g., tablets, troches, lozenges, aqueous, or oily suspensions, dispersible powders or granules, emulsions, hard or soft capsules, or syrups or elixirs. In certain embodiments, said tablets are in the form of matrix tablets in which the release of a soluble active agent(s) is controlled by having the active agent(s) diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi- or multi-layer tablets, made up of two or more distinct layers of granulation compressed together with the individual layers lying one on top of another, with each separate layer containing a different active agent. Bilayer tablets have the appearance of a sandwich since the edge of each layer or zone is exposed. In further embodiments, the compositions comprise the active agent(s) formulated for controlled release in microencapsulated dosage form, in which small droplets of the active agent(s) are surrounded by a coating or a membrane to form particles in the range of a few micrometers to a few millimeters.

Pharmaceutical compositions for oral administration might be formulated so as to inhibit the release of one or both of the active agents in the stomach, i.e., delay the release of one or both of the active agents until at least a portion of the dosage form has traversed the stomach, in order to avoid the acidity of the gastric contents from hydrolyzing the active agent. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylicacid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

In certain embodiments, the invention provides a pharmaceutical composition for oral administration, which is solid and may be in the form of granulate, granules, grains, beads or pellets, mixed and filled into capsules or sachets, or compressed to tablets by conventional methods. In some particular embodiments, the pharmaceutical composition is in the form of a bi- or multilayer tablet, in which each one of the layers comprise one of the two active agents, and the layers are optionally separated by an intermediate, inactive layer, e.g., a layer comprising one or more disintegrants.

Another contemplated formulation is depot systems, based on biodegradable polymers. As the polymer degrades, the active agent(s) is slowly released. The most common class of biodegradable polymers is the hydrolytically labile polyesters prepared from lactic acid, glycolic acid, or combinations of these two molecules. Polymers prepared from these individual monomers include poly (D,L-lactide) (PEA), poly (glycolide) (PGA), and the copolymer poly (D,L-lactide-co-glycolide) (PEG).

Pharmaceutical compositions for oral administration may be prepared according to any method known to the art and may further comprise one or more agents selected from sweetening agents, flavoring agents, coloring agents and preserving agents in order to provide pharmaceutically elegant and palatable preparations. Tablets contain the active agents in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate: granulating and disintegrating agents, e.g., corn starch or alginic acid: binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

Useful dosage forms of the pharmaceutical compositions include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

The pharmaceutical composition of the invention may comprise one or more pharmaceutically acceptable excipients. For example, a tablet may comprise at least one filler, e.g., lactose, ethylcellulose, microcrystalline cellulose, silicified microcrystalline cellulose: at least one disintegrant, e.g., cross-linked polyvinylpyrrolidinone: at least one binder, e.g., polyvinylpyridone, hydroxypropylmethyl cellulose: at least one surfactant, e.g., sodium laurylsulfate: at least one glidant, e.g., colloidal silicon dioxide; and at least one lubricant, e.g., magnesium stearate.

Pharmaceutical compositions for rectal administration may be in any suitable form, e.g., a liquid or gel for injection into the lower bowel by way of the rectum using an enema, or formulated as a suppository, i.e., a solid dosage form for insertion into the rectum.

The pharmaceutical composition of the invention may be in the form of a sterile injectable aqueous or oleagenous suspension, which may be formulated according to the known art using suitable dispersing, wetting or suspending agents. The sterile injectable preparation may also be an injectable solution or suspension in a non-toxic parenterally acceptable diluent or solvent. Acceptable vehicles and solvents that may be employed include, without limiting, water. Ringer's solution, polyethylene glycol (PEG), 2-hydroxypropyl-β-cyclodextrin (HPCD), a surfactant such as Tween-80, and isotonic sodium chloride solution.

Pharmaceutical compositions according to the invention, when formulated for inhalation, may be in any suitable form, e.g., liquid or fine powder, and may be administered utilizing any suitable device known in the art, such as pressurized metered dose inhalers, liquid nebulizers, dry powder inhalers, sprayers, thermal vaporizers, electrohydrodynamic aerosolizers, and the like.

The pharmaceutical compositions of the present invention, as defined in any one of the embodiments above, are useful in preventing, inhibiting, reducing or ameliorating demyelination, thereby more specifically treating a disease, disorder or condition characterized by or associated with demyelination, as defined above.

The pharmaceutical compositions of the invention may be administered, e.g., continuously, daily, twice daily, thrice daily or four times daily, for various duration periods, e.g., weeks, months, years, or decades. The dosages will depend on the state of the patient, and will be determined, from time to time, as deemed appropriate by the practitioner. For example, a physician or veterinarian could start doses of the active agents employed in the pharmaceutical composition at levels lower than required in order to achieve the desired therapeutic effect, and gradually increase the dosage until the desired effect is achieved.

In still another aspect, the present invention relates to a drug combination as defined above, i.e., a combination of a non-iron metal-DFO complex or a pharmaceutically acceptable salt thereof, and an immunomodulatory drug, as defined in any one of the embodiments above, for use in preventing, inhibiting, reducing, or ameliorating demyelination. As disclosed herein, said drug combination may comprise a sub-therapeutic dose of the immunomodulatory drug. i.e., the daily dose of said immunomodulatory drug may be lower than the therapeutically effective dose of said drug when administered alone.

In yet another aspect, the present invention relates to use of a combination of a non-iron metal-DFO complex or a pharmaceutically acceptable salt thereof, and an immunomodulatory drug, as defined in any one of the embodiments above, in the preparation of a pharmaceutical composition for preventing, inhibiting, reducing, or ameliorating demyelination. As disclosed herein, the dose of the immunomodulatory drug used for the preparation of the composition may be either a therapeutic or sub-therapeutic dose.

As previously shown, DFO is capable of abstracting metals such as Fe and Zn from human plasma in vitro (Sooriyaarachchi and Gailer, 2010). It is thus postulated that under physiological conditions, administration of DFO or a pharmaceutically acceptable salt thereof, and metal ions, e.g., Zn- or Ga-ions, from two separate compositions, either concomitantly or sequentially (provided that the interval between administrations of the two components is determined such that at least a major amount of the component first administered is available in the circulation, i.e., not yet excreted, at the time the second component is administered), will result in the formation of a metal-DFO complex, or a pharmaceutically acceptable salt thereof, in situ.

The present invention thus further relates to a method for preventing, inhibiting, reducing or ameliorating demyelination in a subject in need thereof, similar to the method defined above, wherein instead of administering a therapeutically effective amount of a non-iron metal-DFO complex or a pharmaceutically acceptable salt thereof, said subject is administered with amounts of (i) DFO or a pharmaceutically acceptable salt thereof; and (ii) ions of at least one metal other than iron, either concomitantly or sequentially at any order and within a time period not exceeding 36 hours, so as to form in situ upon complexation of said DFO or pharmaceutically acceptable salt thereof and said metal ions, a therapeutically effective amount of said non-iron metal-DFO complex or pharmaceutically acceptable salt thereof, which acts together with the immunomodulatory drug administered to prevent, inhibit, reduce or ameliorate demyelination in said subject.

In certain embodiments, the DFO or pharmaceutically acceptable salt thereof, and the ions of the metal, are administered from two separate pharmaceutical compositions using the same or different administration modes, either concomitantly or sequentially at any order and within a time period not exceeding 36 hours, e.g., within a time period of up to about 2, 4, 6, 8, 10, 12, 14, 16, 18, 20, 22, 24, 26, 28, 30, 32, 34 or 36 hours, such that at least a major amount of the component first administered is available in the circulation at the time the second component is administered, and said metal-DFO complex or pharmaceutically acceptable salt thereof may thus be formed in situ.

Examples of metal ions and immunomodulatory drugs that may be administered according to the method described hereinabove are listed above. In particular embodiments, the metal ions administered are ions of Zn:Ga; or a mixture of Zn and any one of the other non-iron metal ions listed above, e.g., Ga, e.g., wherein the quantitative ratio of the Zn ions to the other non-iron metal ions is in a range of 100:1 to 1:100.

The metal ions administered may be in the form of cations (salts) in any possible valence state (depending on the specific metal), or in complexes with organic compounds such as aromatic and non-aromatic compounds having a heteroatom-containing moiety, e.g., carbonyl compounds, hydroxy compounds, heterocyclic compounds. Non-limiting examples of ligands (mono-, bi-, tridentate-, etc.) forming metal complexes are acetate, gluconate and acetylacetone, tris(2-aminoethyl)amine, crown ethers, porphyrins, alkyl phosphates such as dialkyldithiophosphate, and heterocycles such as terpyridine, pyrithione and metallocenes.

For example, zinc ions may be present in the form of a zinc salt, e.g., ZnCl₂, or in complexes such as zinc acetate, zinc crown ether, Zn-porphyrin/crown ether conjugate, zinc protoporphyrin, zinc chlorophyll and bacteriochlorophyll, monomeric zinc dialkyldithiophosphate, zinc acetylacetone (trimer; Zm/AcAcy), zinc terpyridine (tridentate: [Zn(Terpy)Cl₂]), zinc tris(2-aminoethyl)amine, carbonic anhydrase (Zn metalloenzyme), glutamate carboxypeptidase II (Zn metalloenzyme), organozinc compounds such as diethylzinc (I) and decamethyldizincocene (II), Zinc gluconate, and zinc pyrithione. Gallium ions may be present in the form of a gallium salt, e.g., GaCl₃.

As defined above, the non-iron metal-DFO complex or pharmaceutically acceptable salt thereof formed in situ, and the immunomodulatory drug administered may be at any quantitative ratio, e.g., at a quantitative ratio in a range of 100:1 to 1:100 as defined above.

In a further aspect, the present invention provides a kit comprising (i) either a pharmaceutical composition A comprising a non-iron metal-DFO complex or a pharmaceutically acceptable salt thereof; or pharmaceutical compositions B and C, wherein pharmaceutical composition B comprises DFO or a pharmaceutically acceptable salt thereof, and pharmaceutical composition C comprises ions of a non-iron metal: (ii) a pharmaceutical composition D comprising an immunomodulatory drug; and (iii) instructions to administer cither (a) pharmaceutical compositions A and D, either concomitantly or sequentially at any order and within a time period not exceeding 36 hours, e.g., within a time period of up to about 2, 4, 6, 8, 10, 12, 14, 16, 18, 20, 22, 24, 26, 28, 30, 32, 34 or 36 hours, to thereby prevent, inhibit, reduce, or ameliorate demyelination, thus more particularly treat a disease, disorder or condition characterized by or associated with demyelination: or (b) pharmaceutical compositions B, C and D, either concomitantly or sequentially at any order and within a time period not exceeding 36 hours, so as to form in situ, upon complexation of said DFO or pharmaceutically acceptable salt thereof and said metal ions, a non-iron metal-DFO complex or a pharmaceutically acceptable salt thereof, to thereby prevent, inhibit, reduce, or ameliorate demyelination, thus more particularly treat a disease, disorder or condition characterized by or associated with demyelination.

In certain embodiments, the non-iron metal-DFO complex comprised within pharmaceutical composition A is the zinc-DFO complex, gallium-DFO complex, manganese-DFO complex, copper-DFO complex, aluminum-DFO complex, vanadium-DFO complex, indium-DFO complex, chromium-DFO complex, gold-DFO complex, silver-DFO complex, or platinum-DFO complex, a lanthanide-DFO complex, or a mixture thereof in any quantitative ratio. In other embodiments, the non-iron metal ions comprised within pharmaceutical composition C are ions of zinc, gallium, manganese, copper, aluminum, vanadium, indium, chromium, gold, silver, platinum, a lanthanide, or a mixture thereof in any quantitative ratio. The metal ions may be in the form of cations in any possible valence state, or in complexes with organic compounds such as aromatic and nonaromatic compounds having a heteroatom-containing moiety, as defined above.

In certain embodiments, the non-iron metal-DFO complex comprised within pharmaceutical composition A is Zn-DFO complex, Ga-DFO complex, or a mixture of Zn-DFO complex and any one of the other non-iron metal-DFO complexes listed above, e.g., Ga-DFO complex, e.g., wherein the quantitative ratio of said Zn-DFO complex to the other metal-DFO complex in said mixture is in a range of 100:1 to 1:100; or said pharmaceutical composition C comprises ions of Zn, Ga, or Zn and any one of the other metal ions listed above, e.g., Ga, e.g., wherein the quantitative ratio of the Zn ions to the other metal ions is in a range of 100:1 to 1:100.

In certain embodiments, the amount of the non-iron metal-DFO complex or pharmaceutically acceptable salt thereof comprised in pharmaceutical composition A, or alternatively, the amounts of the DFO complex or pharmaceutically acceptable salt thereof comprised in pharmaceutical composition B, and of the metal ions comprised in pharmaceutical composition C, are determined such that the metal-DFO complex administered or formed in situ, and the immunomodulatory drug, are at any quantitative ratio, e.g., at a quantitative ratio in a range of 100:1 to 1:100 as defined above.

The pharmaceutical compositions contained within the kit of the invention may be formulated, each independently, for any suitable administration route, as defined above.

The kit disclosed herein may comprise each one of the compositions in a ready for use form, e.g., formulated as a liquid for topical, nasal or oral administration, or may alternatively include one or both of the compositions as a solid composition that can be reconstituted with a solvent to provide a liquid oral dosage form. In cases one or more of the compositions are provided in a solid form for reconstitution with a solvent, the kit may further include a reconstituting solvent and instructions for dissolving said solid composition in said solvent prior to administration. Such a solvent should be pharmaceutically acceptable and may be, e.g., water, an aqueous liquid such as phosphate buffered saline (PBS), a non-aqueous liquid, or a combination of aqueous and non-aqueous liquids. Suitable non-aqueous liquids include, but are not limited to, oils, alcohols such as ethanol, glycerin, and glycols such as polyethylene glycol and propylene glycol.

The kit of the present invention, according to any one of the embodiments defined above, is useful for preventing, inhibiting, reducing, or ameliorating demyelination, and thus for treating diseases, disorders, or conditions characterized by or associated with demyelination.

Unless otherwise indicated, all numbers expressing quantities of ingredients and so forth used in the present description and claims are to be understood as being modified in all instances by the term “about”. Accordingly, unless indicated to the contrary, the numerical parameters set forth in this specification and attached claims are approximations that may vary by up to plus or minus 10% depending upon the desired properties sought to be obtained by the present invention.

The invention will now be illustrated by the following non-limiting Examples.

### EXAMPLES

### Example 1. Blood-Brain Barrier (BBB) Penetration

In this study, the ability of the Zn-DFO complex to infiltrate into the brain was examined. Rats were injected intraperitoneally (IP) with increasing amounts of DFO alone or Zn-DFO complex, and the concentration of DFO was measured in their brains, thus monitoring their infiltrability.

In particular, male Sprague-Dawley (SD) rats (300 g average body weight, 3 animals per group) were injected IP with DFO (250, 500, or 1000 mg/kg body weight). For comparison, Zn-DFO solution in freshly prepared saline was injected IP (250, 500, or 1000 mg/kg body weight, which are equivalent to 221, 442, or 883 mg DFO/kg body weight, respectively).

The behavior of the rats was monitored for 90 min following the injection. Then, the animals were euthanized using an injection of ketamine-xylazine, and their hearts, livers, left kidneys, and brains were excised and weighted. Brain samples (300 mg tissue) were washed homogenized in 3 ml Lysis buffer. Tissue homogenates were incubated at 110° C. for 5 min and centrifuged for 5 min at 14,500 relative centrifugal force (RCF). An equal volume of 40% trichloroacetic acid (TCA) solution was added, forming a solution containing 20% TCA, which was vortexed and centrifuged again. The supernatants were transferred into cuvettes, and DFO concentration was spectrophotometrically measured, after addition of 10 mM of ferric iron solution (as a weak complex of iron, ferric-nitrilo-triacetate, at pH=7.4), and scanning the range of λ=380−580 nm. The final concentration was calculated according to the Beer-Lambert law, using εDFO=2460 M⁻¹cm⁻¹: the total amount of DFO per gram tissue and per whole brain were calculated. The results obtained are summarized in Table 1.

TABLE 1
Comparative study of the infiltrability of Zn-DFO and DFO into the rat brain

| | | | | DFO (mg/whole | DFO inti It rated |
|---|---|---|---|---|---|
| | Amount | | DFO (mg/g organ) | organ) | the brain (C) |
| Zn-DFO | 1000 | mg/kg | 0.072 + 0.011 | 0.133 ± 0.010 | 0.014% |
| | 500 | mg/kg | 0.031 + 0.001 | 0.054 + 0.002 | 0.012% |
| | 250 | mg/kg | 0.017 + 0.002 | 0.032 + 0.004 | 0.014% |
| DFO | 1000 | mg/kg | 0.008 + 0.004 | 0.019 + 0.008 | 0.002% |
| | 500 | mg/kg | <0.005 | <0.005 | <0.0015% |
| | 250 | mg/kg | <0.005 | <0.005 | <0.0015% |

*The results are shown as average ± standard error.
‡ The OD was below the detection limit of the instrument (<0.005)

Fifteen min after the administration of 1000 mg/kg Zn-DFO complex, the rats looked passive and apathetic; however, no changes in the rats' behavior could be identified after injection of either 500 mg/kg or 250 mg/kg of Zn-DFO complex. Following injection of DFO alone, for the three doses, no changes in rats' behavior was observed.

The total amount of DFO found in the brain (mg/g), at the end of the experiments, was calculated for all groups—3 in each group, administered with DFO alone (250, 500 and 1000 mg/kg) and 3 administered with Zn-DFO (250, 500 and 1000 mg/kg). The fraction of DFO that had infiltrated into the brain remained nearly constant for the 3 doses of Zn-DFO, with an average of 0.0133%. For the corresponding doses of DFO alone, the value was 0.002% or less (due to the limited sensitivity of the instrument). Thus, the infiltrability of the Zn-DFO complex into the brain is at least 6.8-fold higher than that for the DFO alone.

### Example 2. Experimental Autoimmune Encephalomyelitis—a Model for Multiple Sclerosis (MS)

In this study, the therapeutic effect of treatment with glatiramer acetate (Copaxone®) alone or in combination with Zn-DFO on multiple sclerosis (MS) is tested, using the murine Experimental Autoimmune Encephalomyelitis (EAE) model.

EAE is induced in 8-week-old female C57BL/6 mice by subcutaneous (SC) injection of 125 μg of myelin oligodendrocyte glycoprotein 35-55 peptide (MOG₃₅₋₅₅) emulsified in complete Freund's adjuvant (CFA) containing 5 mg/ml heat-killed Mycobacterium tuberculosis into the left para-lumbar region. Immediately thereafter, and again at 48 h, the mice are inoculated intraperitoneal (IP) with 0.5 ml of pertussis toxin (400 ng). Seven days later, the mice are further challenged with an additional injection of MOG₃₅₋₅₅ peptide in CFA injected into the right para-lumbar region.

TABLE 2
Scale for assessing the severity of MS

| Grade | Clinical sign | Comments |
|---|---|---|
| 0 | No clinical signs | Normal gait, tail moves and can be raised, tail wraps |
| | | around object if mouse is held at the base of the tail |
| 1 | Partially limp tail | Normal gait, tip of the tail droops |
| 2 | Paralyzed tail | Normal gaid, tail droops |
| 3 | Hind limb paresis, | Uncoordinated gait, tail limps, hind limbs respond to |
| | uncoordinated movement | pinching |
| 4 | One hind limb paralyzed | Uncoordinated gait with one hind limb dragging, tail |
| | | limps, one hind limb does not respond to pinch |
| 5 | Both hind limbs paralyzed | Uncoordinated gait with both hind limbs dragging, tail |
| | | limps, both hind limbs do not respond to pinch |
| 6 | Hind limbs paralyzed, | Uncoordinated gait with forelimbs struggle to pull body, |
| | weakness in forelimbs | forelimbs reflex after pinching, tail limps |
| 7 | Hind limbs paralyzed, one | Mouse cannot move, one forelimb responds to pinch, |
| | forelimb paralyzed | tail limps |
| 8 | Hind limbs paralyzed, both | Mouse cannot move, both forelimbs do not respond to |
| | forelimbs paralyzed | pinch, tail limps |
| 9 | Moribund | No movement, altered breathing |
| 10 | Death | |

Mice are treated with SC injection of 200 (μg glatiramer acetate (Copaxone®) per animal in mannitol 4% emulsion on day 0. Part of the animals are continued to be treated with Zn-DFO as IP injections, 2 mg/kg or 6 mg/kg, thrice a week till the end of the experiment (Day 28). Two additional groups of mice are treated with Zn-DFO analogously, but without the preceding injection with glatiramer acetate. The severity of the disease is assessed using the scale shown in Table 2 (Bittner et al. 2014).

It is expected that mice exposed to MOG₃₅₋₅₅, without treatment, will develop the disease during 11-13 days after the first injection, reaching the score of 1-3. The disease will aggravate during the next 3-4 days, reaching a peak value of the clinical signs score of 6-7 on Day 15 (see Table 2). During the next two weeks their condition will improve slightly, to clinical score of 5-6. The treatment either with glatiramer acetate alone or Zn-DFO 2 mg/kg is expected to delay the onset of the disease to Day 14, and the peak clinical score will be reached on Day 17, with a value of 4-5. During the next period the score will be improved to 3-4. The treatment with Zn-DFO 6 mg/kg alone is expected to postpone the onset of the disease to Day 15, and suppress its peak value (Day 19) to 3-4. As the result of treatment with glatiramer acetate combined with Zn-DFO, 6 mg/kg, the disease manifestations are detected on Days 15-16 and the peak clinical score will be less than 3 (Day 19), and then improved to score of 1-2 till the end of the experiment. The control group is not expected to express any clinical sign.

1. A pharmaceutical composition comprising a combination of a metal-desferrioxamine B complex (metal-DFO complex) or a pharmaceutically acceptable salt thereof, wherein said metal is not iron, and an immunomodulatory drug, and a pharmaceutically acceptable carrier.

2. The pharmaceutical composition of claim 1, wherein said metal-DFO complex is the zinc-, gallium-, manganese-, copper-, aluminum-, vanadium-, indium-, chromium-, lanthanum-, gold-, silver-, or platinum-DFO complex, a lanthanide-DFO complex, or a mixture thereof.

3. The pharmaceutical composition of claim 2, wherein said metal-DFO complex is Zn-DFO complex, Ga-DFO complex, or a mixture thereof.

4. The pharmaceutical composition of claim 3, wherein said metal-DFO complex is a mixture of Zn-DFO complex and Ga-DFO complex, and the quantitative ratio of said Zn-DFO complex to said Ga-DFO complex in said mixture is in a range of 100:1 to 1:100.

5. The pharmaceutical composition of claim 1, wherein said immunomodulatory drug is fingolimod, dimethyl fumarate, teriflunomide, glatiramer acetate, ocrelizumab, natalizumab, alemtuzumab, an immuno-enhancing interferon-based drug, or a pharmaceutically acceptable salt thereof.

6. The pharmaceutical composition of claim 1, wherein the quantitative ratio of said metal-DFO complex to said immunomodulatory drug in said combination is in a range of 100:1 to 1:100.

7. The pharmaceutical composition of claim 1, formulated for oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

8. The pharmaceutical composition of claim 1, for preventing, inhibiting, reducing or ameliorating demyelination.

9. The pharmaceutical composition of claim 8, for treatment of multiple sclerosis (MS), neuromyelitis optica (Devic's disease), Balo concentric sclerosis, Schilder's disease, chronic inflammatory demyelinating polyneuropathy, progressive multifocal leukoencephalopathy, Guillain-Barre syndrome, progressive inflammatory neuropathy, acute disseminated encephalomyelitis, optic neuritis, transverse myelitis, adrenoleukodystrophy, or adrenomyeloneuropathy.

10. The pharmaceutical composition of claim 8, for treatment of a disorder or a condition induced by an injury, such as an injury caused by a mechanical force, ischemia, a toxic agent such as an herbicide or pesticide, or hemorrhage.

11. A combination of a metal-desferrioxamine B complex (metal-DFO complex) or a pharmaceutically acceptable salt thereof, wherein said metal is not iron, and an immunomodulatory drug for use in preventing, inhibiting, reducing, or ameliorating demyelination.

12. Use of a combination of a metal-desferrioxamine B complex (metal-DFO complex) or a pharmaceutically acceptable salt thereof, wherein said metal is not iron, and an immunomodulatory medication in the preparation of a pharmaceutical composition for preventing, inhibiting, reducing, or ameliorating demyelination.

13. A kit, comprising:
(i) either a pharmaceutical composition A comprising a metal-desferrioxamine B complex (metal-DFO complex) or a pharmaceutically acceptable salt thereof; or pharmaceutical compositions B and C, wherein pharmaceutical composition B comprises DFO or a pharmaceutically acceptable salt thereof, and pharmaceutical composition C comprises ions of a metal, wherein said metal is not iron;
(ii) a pharmaceutical composition D comprising an immunomodulatory drug; and
(iii) instructions to administer either (a) pharmaceutical compositions A and D, either concomitantly or sequentially at any order and within a time period not exceeding 36 hours; or (b) pharmaceutical compositions B, C and D, either concomitantly or sequentially at any order and within a time period not exceeding 36 hours, so as to form in situ, upon complexation of said DFO or pharmaceutically acceptable salt thereof and said metal ions, a metal-DFO complex or a pharmaceutically acceptable salt thereof, to thereby prevent, inhibit, reduce, or ameliorate demyelination.

14. The kit of claim 13, wherein said metal-DFO complex is the zinc-, gallium-, manganese-, copper-, aluminum-, vanadium-, indium-, chromium-, gold-, silver- or platinum-DFO complex, a lanthanide-DFO complex, or a mixture thereof; or said pharmaceutical composition C comprises ions of zinc, gallium, manganese, copper, aluminum, vanadium, indium, chromium, gold, silver, platinum, a lanthanide, or a mixture thereof.

15. The kit of claim 13, wherein said metal-DFO complex is Zn-DFO complex, Ga-DFO complex, or a mixture thereof; or said pharmaceutical composition C comprises ions of Zn, Ga, or both Zn and Ga.

16. The kit of claim 15, wherein said metal-DFO complex is a mixture of Zn-DFO complex and Ga-DFO complex, and the quantitative ratio of said Zn-DFO complex to said Ga-DFO complex in said mixture is in a range of 100:1 to 1:100; or said pharmaceutical composition C comprises ions of both Zn and Ga, and the quantitative ratio of the Zn ions to the Ga ions is in a range of 100:1 to 1:100.

17. The kit of claim 13, wherein said immunomodulatory drug is fingolimod, dimethyl fumarate, teriflunomide, glatiramer acetate, ocrelizumab, natalizumab, alemtuzumab, an immuno-enhancing interferon-based drug, or a pharmaceutically acceptable salt thereof.
